(19) United States
(12) Patent Application Publication
(10) Pub. No.: US 2022/0331268 A1
(43) Pub. Date: Oct. 20, 2022

(54) TREATMENT OF SKIN DISORDERS WITH TOPICAL COMBINATION COMPOSITIONS COMPRISING TAPINAROF AND AN ADDITIONAL AHR ACTIVATOR

(71) Applicant: Sol-Gel Technologies Ltd., Ness Ziona (IL)

(72) Inventors: Moshe ARKIN, Kfar Shmaryahu (IL); Marcel ZIGHELBOIM, Kiryat Motzkin (IL)

(73) Assignee: Sol-Gel Technologies Ltd., Ness Ziona (IL)

(21) Appl. No.: 17/763,236

(22) PCT Filed: Sep. 24, 2020
(86) PCT No.: PCT/IL2020/051050

§ 371 Date: Mar. 24, 2022

Related U.S. Application Data
(60) Provisional application No. 62/906,278, filed on Sep. 26, 2019.

(51) Int. Cl. A61K 31/05; A61K 9/00; A61K 31/496; A61K 31/235; A61K 31/203; A61K 31/192; A61K 31/4436; A61K 31/519; A61K 31/222; A61K 31/7048; A61K 31/167; A61K 9/48

(52) U.S. Cl. CPC A61K 31/05; A61K 9/0014; A61K 31/496; A61K 31/235; A61K 31/203; A61K 31/192; A61K 31/4436; A61K 31/519; A61K 31/222; A61K 31/7048; A61K 31/167; A61K 9/4816

(57) ABSTRACT

Provided herein is a topical combination composition comprising tapinarof, an additional AhR activator and optionally further comprising at least one additional active agent selected from a retinoid, benzoyl peroxide (BPO), a Janus kinase inhibitor (JAK inhibitor), a corticosteroid of potency class 1-4, an acaricide and combinations thereof. The active agents in the composition of this invention are in encapsulated or non-encapsulated form, according to need. The above compositions are useful for the treatment, prevention or alleviation of a skin disorder and exhibit synergistic and/or additive effects which allow reducing the amounts of the active agents in the compositions.

### FIELD OF THE INVENTION

The present invention, in some embodiments thereof, relates to treatment of a skin disorder by topical administration of a combination composition comprising tapinarof, an additional AhR activator and optionally an additional active agent. The compositions of this invention are useful for the treatment, prevention or amelioration of certain skin disorders and exhibit synergistic and/or additive effects which allow reducing the dose of the active agents in the combination composition.

### BACKGROUND

Skin disorders (skin conditions) vary greatly in symptoms and in severity. They are commonly treated with systemic and/or topical medicaments. Topical medicaments, while not always available, have the advantage of avoiding systemic side-effects. On the other hand, also topical skin disorder treatments are accompanied by undesirable side-effects, especially at high doses.

The present invention provides novel combination compositions and takes aim at minimizing undesirable side-effects.

### SUMMARY OF THE INVENTION

This invention provides a topical combination composition comprising tapinarof, an additional AhR activator and optionally at least one additional active agent selected from at least one retinoid, benzoyl peroxide (BPO), at least one Janus kinase inhibitor (JAK inhibitor), at least one corticosteroid of potency class 1-4, at least one acaricide selected from permethrin, ivermectin and crotamiton and combinations thereof.

The topical combination compositions are suitable for the treatment, prevention or alleviation of a skin disorder and exhibit synergistic and/or additive effects which allow reducing the amounts of the active agents in the compositions. The addition of an additional AhR activator to tapinarof potentiates the tapinarof therapeutic effect.

### DETAILED DESCRIPTION OF THE INVENTION

The present invention provides novel topical combination compositions and methods of treatment, prevention and amelioration of a skin disorder selected from acne, rosacea, plaque psoriasis, scalp psoriasis, flexural/inverse psoriasis, sebopsoriasis, angular cheilitis, tinea, scabies, scaling skin, dermatitis, erythrasma, folliculitis, external vulvar pruritus and genital psoriasis.

Folliculitis is also a side-effect of tapinarof topical treatment and the combination of tapinarof with an additional AhR activator and optionally at least one additional active agent selected from at least one retinoid, benzoyl peroxide (BPO), at least one Janus kinase inhibitor (JAK inhibitor), at least one corticosteroid of potency class 1-4, at least one acaricide selected from permethrin, ivermectin and crotamiton and combinations thereof—results in cancelling or diminishing this side-effect.

Some of the known topical skin disorder treatments use pharmaceutical active agents selected from corticosteroids like halobetasol or desoxymethasone and Vitamin D analogues like calcipotriene or paricalcitol. Combinations of several of the above classes of active agents, like Vitamin D and corticosteroids have been investigated. Most of the known topical treatments of skin disorders in general, and those comprising steroids in particular, present undesirable side-effects.

The topical combination composition of this invention comprises from about 0.25% w/w to about 3.0% w/w tapinarof, from about 0.25% w/w to about 3% w/w of an additional AhR activator and a carrier suitable for topical administration.

Also provided is a topical combination composition further comprising at least one additional active agent selected from about 0.025% w/w to about 0.5% w/w of at least one retinoid, from about 2% w/w to about 10% w/w benzoyl peroxide (BPO), from about 0.1% w/w to about 3.0% w/w at least one Janus kinase inhibitor (JAK inhibitor), from about 0.01% w/w to about 0.25% w/w at least one corticosteroid of potency class 1-4, from about 0.5% to about 5% w/w at least one acaricide selected from permethrin, ivermectin and crotamiton and combinations thereof.

It occurred to the present inventors, that topical combination compositions comprising tapinarof and an additional AhR activator, optionally further comprising at least one additional active agent may allow treatment of skin disorders for longer periods of time, exhibit improved therapeutic effects and also synergistic or additive effects in the treatment of a skin disorder and, as a result, allow using lower dosage of the actives and diminish the product's side-effects (like local irritation and contact dermatitis). The optional addition of at least one additional active agent selected from at least one retinoid, benzoyl peroxide (BPO), at least one Janus kinase inhibitor (JAK inhibitor), at least one corticosteroid of potency class 1-4, at least one acaricide selected from permethrin, ivermectin and crotamiton and combinations thereof further broadens the scope of therapeutic activity of the tapinarof-imidazole active agent combination composition.

Without wishing to be bound by theory, the combination of tapinarof (an AhR agonist, according to Smith S. H. et al, J. of Investigative Dermatology (2017) 137, 2110-2119) with an additional AhR activator and optionally with an additional active agent provides additive and/or synergistic effects, enabling lower concentrations and dosages of the active agents, thus minimizing their side-effects. Both tapinarof and the additional AhR activator have anti-inflammatory and anti-oxidant activities and their combination has better activity than each of its constituents.

In some embodiments, there is provided a topical composition comprising from about 0.25% w/w to about 3.0% w/w tapinarof, from about 0.25% w/w to about 3% w/w of an additional AhR activator and a carrier suitable for topical administration.

In some other embodiments, there is provided a topical combination composition further comprising about 0.025% w/w to about 0.5% w/w of at least one retinoid, from about 2% w/w to about 10% w/w benzoyl peroxide (BPO), from about 0.1% w/w to about 3.0% w/w at least one Janus kinase inhibitor (JAK inhibitor), from about 0.01% w/w to about 0.25% w/w at least one corticosteroid of potency class 1-4, from about 0.5% to about 5% w/w at least one acaricide selected from permethrin, ivermectin and crotamiton and combinations thereof.

The tapinarof-additional AhR activator combination composition of this invention has a double advantage vs. the use of tapinarof or an additional AhR activator as single drugs: on the one hand the additional AhR activator has the potential to alleviate the tapinarof side-effects, especially at high concentrations, and on the other hand the synergistic and/or additive effect may enable using lower active agent amounts. The addition of an additional AhR activator to tapinarof potentiates the tapinarof therapeutic effect.

There is an unmet need for methods of treatment of a skin disorder using tapinarof—additional AhR activator—optional additional active agent topical combination compositions in lower doses, devoid of serious side-effects, which may be used for extended periods of time.

The active agents in the compositions of this invention are detailed below.

### Tapinarof

Tapinarof (3,5-dihydroxy-4-isopropyl-trans-stilbene, benvitimod, GSK2894512) is a first-in-class drug, whose mechanism is not yet fully understood. It is being developed by Glaxo Smith Kline (Stiefel, a GSK company) and Dermavant as a topical drug for treatment of mild to moderate psoriasis and atopic dermatitis. It was shown in both mouse models and in vitro human skin studies to inhibit specific proinflammatory mediators, including interleukin-6 and interleukin-17A, and enhance skin barrier function (J Invest Dermatol. 2017 October; 137[10]:2110-9).

According to Jancin B. (Dermatology News, Nov. 11, 2017), in the above studies, the 1% tapinarof arm showed higher efficacy and had a quicker onset of action than the 0.5% arm or vehicle. The most frequent adverse events associated with tapinarof were folliculitis and contact dermatitis.

Tapinarof, which seems to be a significant advance in psoriasis treatment, presents however higher adverse effects (44.5%) when compared to placebo (20.2%) and calcipotriene (19.5%) as a single drug (Medscape Mar. 5, 2017 report by Frellick M., on Abstr. 5629, Amer. Acad. of Dermatology meeting, Mar. 4, 2017).

The most common tapinarof side-effects (>5%) across all groups treated included folliculitis (9%) and contact dermatitis (AdisInsight “Tapinarof-Dermavant Sciences/Welichem Biotech”, updated 29 Aug. 2019).

There is an unmet need for methods for the treatment of skin disorders using tapinarof topical compositions, devoid of serious side-effects.

The present invention solves the aforementioned side-effects, i.e. by encapsulating tapinarof by a process detailed in Examples 1 and 2 (see also U.S. Pat. No. 9,687,465 and published U.S. Patent Application No. 2018147165 (to Sol-Gel Technologies)).

The tapinarof encapsulation process detailed in Examples 1 and 2 allows the use of tapinarof concentrations equal or higher than 1% w/w with minimal or no side-effects.

In addition, the encapsulation process detailed in Examples 1 and 2 improves the chemical stability of tapinarof in multi-component combination compositions.

The additive or synergistic effects of tapinarof with at least one imidazole active agent and optionally at least one additional active agent allow reducing the amounts of the active agents (including the amount of tapinarof) in the composition of this invention.

Without wishing to be bound by theory, combining tapinarof with an additional AhR activator avoids or diminishes the tapinarof folliculitis side-effect.

Tapinarof is an AhR agonist (Smith S. H. et al, J. of Investigative Dermatology (2017) 137, 2110-2119).

AhR activators (AhR agonists)

### Azole Active Agents

Azole active agents belong to a group of active agents comprising an azole ring and having anti-inflammatory, antifungal and/or antibiotic activity. There are two important groups of azole active agents: imidazoles (having two nitrogen atoms in the azole ring) and triazoles (having three nitrogen atoms in the azole ring).

Imidazoles: the most widely used imidazole active agents are, ketoconazole, clotrimazole, econazole, tioconazole, miconazole and butoconazole. They are administered mainly topically or vaginally.

Triazoles: the triazole active agent group includes itraconazole, fluconazole, terconazole, voriconazole, eficonazole, albaconazole, ravuconazole and isavuconazole and combinations thereof. Itraconazole is presently marketed only for oral use.

Flavonoids: (bioflavonoids): Flavonoids are polyhydroxy polyphenol natural compounds, widely found in fruits and vegetables. This class of compounds has anti-oxidative and anti-inflammatory properties (Panche A. N. et al. J Nutr Sci, 2016; 5: e47).

The flavonoids are selected from pentahydroxyflavonoids, hexahydroxyflavonoids, tetrahydroxyflavonoids, trihydroxyflavonoids, quercetin, apigenin and combinations thereof.

### Retinoids

Retinoids are a class of chemical compounds structurally related to Vitamin A which are effective in the treatment of a number of skin disorders, like acne and psoriasis.

Long-term high intake of retinoids causes a number of side-effects.

The retinoids belong to several generations and the main members of this group are tretinoin, adapalene and tazarotene.

Tretinoin, a first-generation retinoid (also known as all-trans retinoic acid or ATRA) is used topically for the treatment of acne.

Adapalene, a third-generation retinoid, is used topically for the treatment of mild to moderate acne.

Tazarotene (marketed as Tazorac, Avage, Zorac and Fabior) is a third-generation prescription topical retinoid sold as a cream, gel, or foam. Tazarotene exhibits side-effects like itching, redness, burning and stinging, especially on long-term treatment.

Treatment for extended periods of time is important, for example for the therapy of a chronic skin disorder, and the composition of this invention allows long-term treatment.

### Benzoyl Peroxide (BPO)

BPO topical gel (alone or in combination with another active agent selected from adapalene, clindamycin, erythromycin) is used the topical treatment of acne.

Topical BPO treatment is accompanied by side-effects like skin irritation, itching, peeling and reddened skin. The compositions of the instant disclosure reduce these BPO side-effects.

Due to its peroxide chemical structure, BPO presents several problems:

a. BPO is a strong oxidant, which may compromise the chemical stability of the other active agents in the combination compositions of this invention and
b. Side-effects like skin irritation, itching, peeling and reddened skin.

The BPO-comprising compositions of this invention use micronized BPO as raw-material and several solutions to the above side-effects:

(i) BPO encapsulation according to Examples 1 and 2, U.S. Pat. No. 9,687,465 and published U.S. Patent Application No. 2018147165 (to Sol-Gel Technologies), whose contents are enclosed herein in their entirety, thus protecting the other active agents in a BPO-combination composition from the BPO oxidation effect and minimizing the BPO skin irritation side-effect.
(ii) Topical application of the BPO-comprising compositions of this invention to the affected skin area of a subject in need thereof as two separate compositions (simultaneously or sequentially in either order) to be mixed on the subject's skin, the first composition comprising tapinarof, at least one AhR activator and a carrier suitable for topical administration and the second composition comprising benzoyl peroxide and a carrier suitable for topical administration (see Example 7). Due to this mode of administration, BPO does not compromise the chemical stability of the other active agents in the combination compositions of this invention. The administration can be done for example by applying the two separate compositions to the affected area of the skin of a subject in need thereof from two application syringes or from a dual chamber application syringe, simultaneously or sequentially in either order. In a preferred product according to the invention, the first and second compositions are respectively filled in the suitable ratio in the two chambers of a dual chamber dispensing system of the type described in EP-A-0644129 and U.S. Pat. No. 5,356,040, the contents of which are incorporated herein by reference. Such a system has two side-by-side chambers, each equipped with a dispensing valve; these are operated by adjacent actuators so as to dispense the formulations either simultaneously or separately as desired. Suitable dispensing systems having chambers which are each capable of holding about 15 ml of composition, are available from Maplast S. r. 1., Via Pasublo 3, Tradate 21049 VA, Italy. The respective dimensions of the dispenser means may be chosen to provide dispensing of the respective compositions in a predetermined ratio (see Example 7).

### Janus Kinase Inhibitors (JAK Inhibitors)

JAK inhibitors are a class of drugs interfering with the JAK-STAT signaling pathway by inhibiting at least one of the Janus kinase enzymes JAK1, JAK2, JAK 3 or TYK2. Some JAK inhibitors inhibit all the above enzymes and are therefore named pan-JAK inhibitors.

The at least one JAK inhibitor in the compositions of this invention is selected from a JAK1 inhibitor, a JAK2 inhibitor, a JAK3 inhibitor, a TYK2 inhibitor a pan-JAK inhibitor and combinations thereof.

The preferred at least one JAK inhibitor in the compositions of this invention is selected from tofacitinib, abrocitinib, delgocitinib, ruxolitinib and combinations thereof.

### Corticosteroids

The tapinarof-imidazole active agent composition of this invention may further comprise a low corticosteroid amount of from about 0.01% w/w to about 0.25% w/w at least one corticosteroid of potency class 1-4, approved for topical use.

The optional corticosteroid in the compositions may be superpotent (Class 1) or potent (Class 2). Alternatively, the steroid may be of lower potency (Class 3-4), thus minimizing the steroid side-effects, such as the risk of pituitary suppression.

According to the “Topical steroid potency chart” of the National Psoriasis Foundation (NPF), the various marketed topical drugs comprising steroids belong to the following potency classes, according to the steroid and the topical drug strength. Due to the different topical drug strength, drugs of different strengths and/or different dosage forms may belong to more than one steroid class.

The percentages in parentheses are the steroid strengths for the FDA-approved topical steroid compositions of potency classes 1-4.

Class 1—superpotent, comprising 7 steroids: clobetasol propionate (0.05%), flurandrenolide (0.05%), betamethasone dipropionate (0.05%), diflorasone diacetate (0.05%), desoxymethasone, and fluocinonide (0.1%).

Class 2—potent, comprising 6 steroids: betamethasone dipropionate (0.05%), mometasone furoate (0.1%), diflorasone diacetate (0.05%), halcinonide (0.1%), fluocinonide (0.05%), desoxymethasone (0.05%-0.25%).

Class 3—upper mid-strength, comprising 3 steroids: fluticasone propionate (0.005%), fluocinonide (0.05%) and betamethasone valerate (0.12%).

Class 4—mid-strength, comprising 6 steroids: flurandrenolide (0.05%), mometasone furoate (0.1%), triamcinolone acetonide (0.1%), fluocinolone acetonide (0.03%), desoxymethasone (0.05%) and hydrocortisone valerate (0.2%).

In some embodiments, there is provided a topical composition comprising from about 0.25% w/w to about 3.0% w/w tapinarof, from about 0.25% w/w to about 3% w/w at least one additional AhR activator, from about 0.01% w/w to about 0.25% w/w at least one corticosteroid of potency class 1-4 and a carrier suitable for topical administration.

### Acaricides

Some of the skin disorders treated with the compositions of this invention are caused inter alia by parasites like ticks and mites. For example, rosacea is caused i.a. by Demodex folliculorum.

The acaricide activity of tapinarof is augmented by addition of an acaricide due to an additive or synergistic effect, allowing to reduce the amounts of the active agents in the composition.

The preferred at least one acaricide in the compositions of this invention is selected from permethrin, ivermectin, crotamiton and combinations thereof.

The skin disorders treated with the compositions of this invention are detailed below.

### Acne

Acne is a chronic inflammatory disease of the pilosebaceous unit resulting from androgen-induced increased sebum production, altered keratinization, inflammation, and bacterial colonisation of hair follicles on the face, neck, chest, and back by Propionibacterium acnes. Although early colonisation with P. acnes and family history might have important roles in the disease, exactly what triggers acne and how treatment affects the course of the disease remain unclear (Williams H. C. et al., The Lancet, Vol. 379. January 2012, pp. 361-372).

There is no ideal treatment for acne. Good quality evidence on comparative effectiveness of common topical and systemic acne therapies is scarce. Topical therapies including benzoyl peroxide, retinoids, and antibiotics when used in combination usually improve control of mild to moderate acne, but suffer from side-effects. Treatment with combined oral contraceptives can help women with acne. Patients with more severe inflammatory acne usually need oral antibiotics combined with topical benzoyl peroxide to decrease antibiotic-resistant organisms.

Oral isotretinoin is the most effective therapy and is used early in severe disease, although its use is limited by teratogenicity and other side-effects.

The treatment of acne with the composition of this invention allows using lower amounts of active agents and therefore reduced side-effects, due to the additive and/or synergistic effects.

### Rosacea (Acne Rosacea)

Rosacea is a chronic skin disease that affects more than 16 million Americans. The cause of rosacea is still unknown, and there is no cure. However, research has allowed doctors to find ways to treat the condition by minimizing its symptoms (Cynthia Cobb, Healthline May 21, 2018).

There are four subtypes of rosacea. Each subtype has its own set of symptoms. It is possible to have more than one subtype of rosacea at a time.

Rosacea's typical symptom is small, red, pus-filled bumps on the skin that are present during flare-ups. Typically, rosacea affects only skin on your nose, cheeks, and forehead.

Flare-ups often occur in cycles. This means that you will experience symptoms for weeks or months at a time, the symptoms will go away, and then return.

The four types of rosacea are:

Subtype one, known as erythematotelangiectatic rosacea (ETR), is associated with facial redness, flushing, and visible blood vessels.

Subtype two, papulopustular (or acne) rosacea, is associated with acne-like breakouts, and often affects middle-aged women.

Subtype three, known as rhinophyma, is a rare form associated with thickening of the skin on your nose. It usually affects men and is often accompanied by another subtype of rosacea.

Subtype four is known as ocular rosacea, and its symptoms are centered on the eye area.

### Psoriasis

Psoriasis is an autoimmune disease, characterized by typically red, scaly patches of skin. There are five main types of psoriasis: plaque, guttate, flexural/inverse, pustular, and erythrodermic. Psoriasis vulgaris is the most common form of psoriasis.

Flexural/Inverse psoriasis is a rare form of psoriasis which is also known as flexural or intertriginous psoriasis. This subtype of psoriasis can occur in any area where two skin surfaces meet. Classically the skin of the groin region, armpits and genitals are affected. In these regions the skin appears red, shiny, and moist, with clear borders, and can sometimes crack in the centre. This rare form of psoriasis accounts for 3-7% of people with psoriasis. A small Chinese study found that the average age of onset for inverse psoriasis is 28.9 years. Occasionally people with another subtype of psoriasis known as pustular psoriasis go on to develop inverse psoriasis.

Recent guidelines from the National Psoriasis Foundation recommend the use of low to moderate strength corticosteroids for flare ups of this type of psoriasis and calcipotriene and either tacrolimus or pimecrolimus (e.g. Elidel) for treatment of inverse psoriasis in the long term.

Though a number of skin disorders treatments are available, most treatments bring about symptom alleviation or remission rather than complete cure.

### Topical Tapinarof Combination Compositions

Provided herein are compositions, combinations, kits and articles of manufacture that include tapinarof in combination with at least one AhR activator and optionally at least one additional active agent, for treating a skin disorder selected from acne, rosacea, plaque psoriasis, flexural/inverse psoriasis, scalp psoriasis, sebopsoriasis, angular cheilitis, tinea, scabies, scaling skin, dermatitis, erythrasma, folliculitis, external vulvar pruritus and genital psoriasis.

The compositions, combinations and articles of manufacture can be administered using a variety of routes such as topical application or transdermal application. The preferred route is the topical route and the preferred formulations are the cream, the gel and the lotion.

Therapeutically effective amounts of tapinarof, at least one AhR activator and optionally an additional active agent are mixed with a suitable pharmaceutical carrier or vehicle suitable and optionally with an anti-oxidant for topical use, for the treatment, prevention or alleviation of the symptoms manifested by a skin disorder.

Tapinarof, at least one additional AhR activator and optionally an additional active agent in the combination compositions are included in an amount effective for treating, preventing or alleviating the skin disorder symptoms. The concentration of the active agents in the composition will depend on absorption, inactivation, excretion rates of the active compound, the synergistic or additive effects, the dosage schedule, and amount administered as well as other factors known to those of skill in the art. Generally, the dosages and concentrations will be lower, typically at least about or at 5 to 10% lower but up to about or at 15, 20, 25, 30, 35, 40, 50, 90 or 95% lower than the amount of tapinarof, an additional AhR activator and optionally an additional active agent in the marketed single drug currently administered for the treatment of a skin disorder. The dosage and regimen of administration may be determined by dose finding studies, as known in the art.

Exemplary dosages, strengths and concentrations of tapinarof in the tapinarof-AhR activator or tapinarof—AhR activator—additional active agent combination compositions administered topically, can be in the range of from about or at 0.1%, 0.25%, 0.5%, 1%, 2% or 3% w/w. Typical strengths in the topical combination compositions of this invention are 0.25%, 0.5% or 1% w/w tapinarof.

TABLE 1
Exemplary Combinations of Active Agents Classes

| Act. Agent | | | | | | | |
|---|---|---|---|---|---|---|---|
| Class/Comb. | TAP | AhRa | RET | BPO | JAKi | CORT | AC |
| TAP/IMID | + | + | | | | | |
| TAP/IMID/RET | + | + | + | | | | |
| TAP/IMID/BPO | + | + | | + | | | |
| TAP/IMID/JAKi | + | + | | | + | | |
| TAP/IMID/CORT | + | + | | | | + | |
| TAP/IMID/AC | + | + | | | | | + |

Legend: Tapinarof (TAP), Additional AhR activator (AhRa), Retinoid (RET), Benzoyl peroxide (BPO), JAK Inhibitor (JAKi), Acaricide (AC)

TABLE 2
Exemplary Combinations of Specific Active Agents

| Act. Agent | | | | | | | |
|---|---|---|---|---|---|---|---|
| Class/Comb. | TAP | AhRa | RET | BPO | JAKi | CORT | AC |
| TAP/ITRA | + | + | | | | | |
| TAP/KETO | + | + | | | | | |
| TAP/ITRA/TRET | + | + | + | | | | |
| TAP/ITRA/ADA | + | + | + | | | | |
| TAP/ITRA/TAZ | + | + | + | | | | |
| TAP/ITRA/BPO | + | + | | + | | | |
| TAP/ITRA/TOFA | + | + | | | + | | |
| TAP/ITRA/ABRO | | | | | + | | |
| TAP/ITRA/DELG | | | | | + | | |
| TAP/ITRA/RUXO | | | | | + | | |
| TAP/ITRA/HAL | + | + | | | | + | |
| TAP/ITRA/PER | + | + | | | | | + |
| TAP/ITRA/IVER | + | + | | | | | + |
| TAP/ITRA/CROT | + | + | | | | | + |

Legend: Tapinarof (TAP), Additional AhR activator (AhRa), Retinoid (RET), Benzoyl peroxide (BPO), JAK Inhibitor (JAKi), Acaricide (AC). Itraconazole (ITRA), Ketoconazole (KETO), Tretinoin (TRET), Adapalene (ADA), Tazarotene (TAZ), Tofacitinib (TOFA), Abrocitinib (ABRO), Delgocitinib (DELG), ruxolitinib (RUXO) Halobetasol (HAL), Permethrin (PER), Ivermectin (IVER), Crotamiton (CROT).

### Exemplary Active Agents' Combination Compositions 1-20 (See Also Tables 1 and 2 Above and Examples 1-50)

### The Active Agents in the Combination Compositions 1-20 May be Encapsulated (See Examples 1-7) or Non-Encapsulated (Examples 8-10)

1. A topical composition comprising from about 0.25% w/w to about 3.0% w/w tapinarof, from about 0.25% w/w to about 3% w/w at least one additional AhR activator selected from an azole active agent selected from an imidazole selected from ketoconazole, clotrimazole, econazole, tioconazole, miconazole and butoconazole, a triazole selected from itraconazole, fluconazole, terconazole, voriconazole, eficonazole, albaconazole, ravuconazole and isavuconazole, a flavonoid selected from a pentahydroxyflavonoid, a hexahydroxyflavonoid, a tetrahydroxyflavonoid, a trihydroxyflavonoid, quercetin and apigenin and combinations thereof and a carrier suitable for topical administration in the treatment, prevention or alleviation of a skin disorder selected from acne, rosacea, plaque psoriasis, scalp psoriasis, flexural/inverse psoriasis, sebopsoriasis, angular cheilitis, tinea, scabies, scaling skin, dermatitis, erythrasma, folliculitis, external vulvar pruritus and genital psoriasis.

2. A topical composition comprising from about 0.25% w/w to about 3.0% w/w tapinarof, from about 0.25% w/w to about 3% w/w itraconazole and a carrier suitable for topical administration in the treatment, prevention or alleviation of a skin disorder.

3. A topical composition comprising from about 0.25% w/w to about 3.0% w/w tapinarof, from about 0.25% w/w to about 3% w/w ketoconazole and a carrier suitable for topical administration in the treatment, prevention or alleviation of a skin disorder.

4. A topical composition comprising from about 0.25% w/w to about 3.0% w/w tapinarof, from about 0.25% w/w to about 3% w/w at least one additional AhR activator selected from an imidazole active agent, a triazole active agent, a flavonoid and combinations thereof, from about 0.025% w/w to about 0.5% w/w of at least one retinoid selected from tretinoin, adapalene and tazarotene and a carrier suitable for topical administration in the treatment, prevention or alleviation of a skin disorder selected from acne, rosacea, plaque psoriasis, flexural/inverse psoriasis, scalp psoriasis, sebopsoriasis, angular cheilitis, tinea, scabies, scaling skin, dermatitis, erythrasma, folliculitis, external vulvar pruritus and genital psoriasis.

5. A topical composition comprising from about 0.25% w/w to about 3.0% w/w tapinarof, from about 0.25% w/w to about 3% w/w itraconazole, from about 0.025% w/w to about 0.5% w/w tretinoin and a carrier suitable for topical administration in the treatment, prevention or alleviation of a skin disorder.

6. A topical composition comprising from about 0.25% w/w to about 3.0% w/w tapinarof, from about 0.25% w/w to about 3% w/w itraconazole, from about 0.025% w/w to about 0.5% w/w adapalene and a carrier suitable for topical administration in the treatment, prevention or alleviation of a skin disorder.

7. A topical composition comprising from about 0.25% w/w to about 3.0% w/w tapinarof, from about 0.25% w/w to about 3% w/w itraconazole, from about 0.025% w/w to about 0.5% w/w tazarotene and a carrier suitable for topical administration in the treatment, prevention or alleviation of a skin disorder.

8. A topical composition comprising from about 0.25% w/w to about 3.0% w/w tapinarof, from about 0.25% w/w to about 3% w/w at least one additional AhR activator selected from an azole active agent selected from an imidazole selected from ketoconazole, clotrimazole, econazole, tioconazole, miconazole and butoconazole, a triazole selected from itraconazole, fluconazole, terconazole, voriconazole, eficonazole, albaconazole, ravuconazole and isavuconazole and combinations thereof and a flavonoid, from about 2% w/w to about 10% w/w benzoyl peroxide (BPO) and a carrier suitable for topical administration in the treatment, prevention or alleviation of a skin disorder selected from acne, rosacea, plaque psoriasis, flexural/inverse psoriasis, scalp psoriasis, sebopsoriasis, angular cheilitis, tinea, scabies, scaling skin, dermatitis, erythrasma, folliculitis, external vulvar pruritus and genital psoriasis.

9. A topical composition comprising from about 0.25% w/w to about 3.0% w/w tapinarof, from about 0.25% w/w to about 3% w/w itraconazole, from about 2% w/w to about 10% w/w benzoyl peroxide (BPO) and a carrier suitable for topical administration in the treatment, prevention or alleviation of a skin disorder.

10. A topical composition comprising from about 0.25% w/w to about 3.0% w/w tapinarof, from about 0.25% w/w to about 3% w/w at least one additional AhR activator selected from an azole active agent selected from an imidazole selected from ketoconazole, clotrimazole, econazole, tioconazole, miconazole and butoconazole, a triazole selected from itraconazole, fluconazole, terconazole, voriconazole, eficonazole, albaconazole, ravuconazole and isavuconazole and combinations thereof and a flavonoid, from about 0.1% w/w to about 3.0% w/w at least one Janus kinase inhibitor (JAK inhibitor) selected from tofacitinib, abrocitinib, delgocitinib, ruxolitinib and combinations thereof and a carrier suitable for topical administration in the treatment, prevention or alleviation of a skin disorder selected from acne, rosacea, plaque psoriasis, flexural/inverse psoriasis, scalp psoriasis, sebopsoriasis, angular cheilitis, tinea, scabies, scaling skin, dermatitis, erythrasma, folliculitis, external vulvar pruritus and genital psoriasis.

11. A topical composition comprising from about 0.25% w/w to about 3.0% w/w tapinarof, from about 0.25% w/w to about 3% w/w itraconazole, from about 0.1% w/w to about 3.0% w/w tofacitinib and a carrier suitable for topical administration in the treatment, prevention or alleviation of a skin disorder.

12. A topical composition comprising from about 0.25% w/w to about 3.0% w/w tapinarof, from about 0.25% w/w to about 3% w/w itraconazole, from about 0.1% w/w to about 3.0% w/w abrocitinib and a carrier suitable for topical administration in the treatment, prevention or alleviation of a skin disorder.

13. A topical composition comprising from about 0.25% w/w to about 3.0% w/w tapinarof, from about 0.25% w/w to about 3% w/w itraconazole, from about 0.1% w/w to about 3.0% w/w delgocitinib and a carrier suitable for topical administration in the treatment, prevention or alleviation of a skin disorder.

14. A topical composition comprising from about 0.25% w/w to about 3.0% w/w tapinarof, from about 0.25% w/w to about 3% w/w itraconazole, from about 0.1% w/w to about 3.0% w/w ruxolitinib and a carrier suitable for topical administration in the treatment, prevention or alleviation of a skin disorder.

15. A topical composition comprising from about 0.25% w/w to about 3.0% w/w tapinarof, from about 0.25% w/w to about 3% w/w at least one additional AhR activator selected from an azole active agent selected from an imidazole selected from ketoconazole, clotrimazole, econazole, tioconazole, miconazole and butoconazole, a triazole selected from itraconazole, fluconazole, terconazole, voriconazole, eficonazole, albaconazole, ravuconazole and isavuconazole and combinations thereof and a flavonoid, from about 0.01% w/w to about 0.25% w/w at least one corticosteroid of potency class 1˜4 and a carrier suitable for topical administration in the treatment, prevention or alleviation of a skin disorder selected from acne, rosacea, plaque psoriasis, flexural/inverse psoriasis, flexural/inverse psoriasis, scalp psoriasis, sebopsoriasis, angular cheilitis, tinea, scabies, scaling skin, dermatitis, erythrasma, folliculitis, external vulvar pruritus and genital psoriasis.

16. A topical composition comprising from about 0.25% w/w to about 3.0% w/w tapinarof, from about 0.25% w/w to about 3% w/w itraconazole, from about 0.01% w/w to about 0.25% w/w halobetasol and a carrier suitable for topical administration in the treatment, prevention or alleviation of a skin disorder.

17. A topical composition comprising from about 0.25% w/w to about 3.0% w/w tapinarof, from about 0.25% w/w to about 3% w/w at least one additional AhR activator selected from an azole active agent selected from an imidazole selected from ketoconazole, clotrimazole, econazole, tioconazole, miconazole and butoconazole, a triazole selected from itraconazole, fluconazole, terconazole, voriconazole, eficonazole, albaconazole, ravuconazole and isavuconazole and combinations thereof and a flavonoid, from about 0.5% to about 5% w/w at least one acaricide selected from permethrin, ivermectin and crotamiton and combinations thereof and a carrier suitable for topical administration in the treatment, prevention or alleviation of a skin disorder selected from acne, rosacea, plaque psoriasis, flexural/inverse psoriasis, scalp psoriasis, sebopsoriasis, angular cheilitis, tinea, scabies, scaling skin, dermatitis, erythrasma, folliculitis, external vulvar pruritus and genital psoriasis.

18. A topical composition comprising from about 0.25% w/w to about 3.0% w/w tapinarof, from about 0.25% w/w to about 3% w/w itraconazole, from about 0.5% to about 5% w/w permethrin and a carrier suitable for topical administration in the treatment, prevention or alleviation of a skin disorder.

19. A topical composition comprising from about 0.25% w/w to about 3.0% w/w tapinarof, from about 0.25% w/w to about 3% w/w itraconazole, from about 0.5% to about 5% w/w ivermectin and a carrier suitable for topical administration in the treatment, prevention or alleviation of a skin disorder.

20. A topical composition comprising from about 0.25% w/w to about 3.0% w/w tapinarof, from about 0.25% w/w to about 3% w/w itraconazole, from about 0.5% to about 5% w/w crotamiton and a carrier suitable for topical administration in the treatment, prevention or alleviation of a skin disorder.

The frequency of administration of the composition of this invention can be determined empirically. Exemplary frequencies are once daily, twice daily, weekly, bi-weekly or monthly. Typical administration frequencies of the topical combination compositions of this invention are once daily and twice daily.

Dosage frequencies can be gradually attenuated over time and maintained at a steady dose suitable for long-term—six months, 1 year, 5 years, 10 years or more, up to lifelong administration to control the symptoms of the skin disorder. For example, dosage administration can begin at from twice a day, to once a day, to two times a week, to once a week, to once every two weeks or less frequent than once every two weeks.

Pharmaceutical carriers or vehicles suitable for preparation of the compositions provided herein include any such carriers known to those skilled in the art to be suitable for the particular mode of administration.

In some embodiment, the compositions of this invention comprises a carrier, a solvent, an emollient, a surfactant, an anti-oxidant, a chelating agent, a gelling agent, a wetting agent, a penetration enhancer, a preservative, an anti-oxidant, a buffer or any combination thereof.

In some embodiments, the compositions of this invention comprise a penetration enhancer. In another embodiment, the penetration enhancer is selected from the group consisting of dimethylsulfoxide (DMSO), diethylene glycol monoethyl ether (Transcutol), methylsulfonylmethane (MSM), oleic acid, oleyl alcohol, propylene glycol, dimethyl isosorbide, isopropyl myristate, diethyl sebacate, ethanol, isopropyl alcohol, a polyethylene glycol, hexylene glycol, glycofurol and any combination thereof.

In some embodiments, the compositions of this invention comprise an emollient. In another embodiment, the emollient is selected from the group consisting of castor oil, mineral oil, vegetable oil, soybean oil, shea butter, cocoa butter, paraffin, beeswax, oleic acid, squalene, cetyl alcohol, isopropyl myristate, urea, glycerol, propylene glycol, lactic acid and any combination thereof.

In some embodiments, the compositions of this invention comprise a chelating agent. In another embodiment, the chelating agent is selected from the group consisting of EDTA deferoxamine, deferiprone, deferasirox, dimercaptosuccinic acid (succimer) triethylenetetramine (trientine) and any combination thereof.

In some embodiments, the compositions of this invention comprise an anti-oxidant. In another embodiment, the anti-oxidant is selected from the group consisting of butylated hydroxytoluene (BHT), parabens, propyl gallate, ascorbic acid, flavones, flavanones, flavonols, stilbenoids, gallic acid, cinnamic acid, ellagic acid, salicylic acid, curcumin, eugenol, citric acid and any combination thereof.

In some embodiments, the compositions of this invention comprise a solvent. In another embodiment the solvent is selected from the group consisting of propylene glycol, diethylene glycol monoethyl ether (Transcutol), dimethylsulfoxide (DMSO), diethyl sebacate, polyethylene glycol, oleyl alcohol, isosorbide dimethyl ether, ethanol, isopropyl alcohol, isopropyl myristate, oleic acid, hexylene glycol, glycerin, glycofurol and any combination thereof.

In some embodiments, the compositions of this invention comprise a preservative. In another embodiment the preservative is selected from the group consisting of benzoic acid, methylparaben, chlorocresol, Phenoxyethanol, propyl paraben, benzalkonium chloride, benzyl alcohol, imidazolidinyl urea, sodium benzoate, sorbic acid, thimerosal sorbic acid, sodium sorbate, sodium sulfite, ethanol, tocopherols and any combination thereof.

In some embodiments, the compositions of this invention comprise a buffer. In another embodiment, the buffer is selected from the group consisting of citric acid, phosphoric acid, acetic acid, tartaric acid, glutamic acid, aspartic acid, malic acid, succinic acid, fumaric acid, salt thereof, and any combination thereof.

In some embodiments, the compositions of this invention comprise a gelling agent. In another embodiment, the gelling agent is selected from the group consisting of carbomer homopolymer type A (Carbopol®981, Carbopol®980), Sepineo P600, Natrosol hydroxyethylcellulose, starch, pectin, gelatin, agar, alginic acid and salts thereof, Carbomer® 934, carboxymethylcellulose, hydroxypropylmethylcellulose and any combination thereof.

In some embodiments, the compositions of this invention comprise a surfactant. In another embodiment, the surfactant is selected from the group consisting of carbomer copolymer type B (Pemulen®TR-1), sorbitan monooleate (Span80), polysorbate 80 (Tween 80), polysorbate 20, polysorbate 60 (Tween 60), sodium dodecylsulfate, cetearyl alcohol, dioctyl sodium sulfosuccinate, glyceryl oleate, glyceryl stearate, poloxamers and any combination thereof.

In some embodiments, the compositions of this invention comprise a wetting agent. In another embodiment, the wetting agent is selected from the group consisting of PEG 400, glycerin, poloxamers, sorbitan monooleate (Span80), polysorbate 80 (Tween 80), polysorbate 20, polysorbate 60 (Tween 60), benzalkonium chloride, sodium dodecylsulfate and any combination thereof.

The resulting composition may be a cream, a lotion, a gel, an ointment, an emulsion, a solution, a suspension, an elixir, a tincture, a paste, a foam, an aerosol, a spray, a patch, a transdermal patch, a powder, a shake lotion, a solid, a sponge, a tape, a shampoo and an applicator syringe (for intradermal injection), or any other formulation suitable for topical administration. The preferred compositions are the cream, the gel and the lotion.

Pharmaceutical carriers or vehicles suitable for administration of the compounds provided herein include any such carriers known to those skilled in the art to be suitable for the particular mode of administration. In addition, the compounds may be formulated as the sole pharmaceutically active ingredient in the composition or may be combined with other active ingredients. The active agents are included in the carrier in an amount sufficient to exert a therapeutically useful effect i.e., amelioration of the symptoms of the skin disorder, with minimal or no toxicity or other side effects. Generally, emollient or lubricating vehicles that help hydrate the skin are more preferred than volatile vehicles, such as ethanol, that dry the skin. Examples of suitable bases or vehicles for preparing compositions for use with human skin are petrolatum, petrolatum plus volatile silicones, lanolin, cold cream and hydrophilic ointment.

Suitable pharmaceutically and dermatologically acceptable vehicles for topical application include lotions, creams, solutions, gels, tapes and the like. Generally, the vehicle is either organic in nature or an aqueous emulsion and capable of having the selected compound or compounds, which may be micronized, dispersed, suspended or dissolved therein. The vehicle may include pharmaceutically-acceptable emollients, moisturizers, including lactic acid, ammonium lactate and urea, skin penetration enhancers, coloring agents, fragrances, emulsifiers, thickening agents, vegetable oils, essential oils, zinc oxide and solvents.

### Methods of Treatment

According to an aspect of the invention, there is provided a method of treatment of a skin disorder selected from acne, rosacea, plaque psoriasis, flexural/inverse psoriasis, scalp psoriasis, sebopsoriasis, angular cheilitis, tinea, scabies, scaling skin, dermatitis, erythrasma, folliculitis, external vulvar pruritus and genital psoriasis. by treatment of a subject in need thereof with a combination composition of tapinarof-additional AhR activator or tapinarof-additional AhR activator-additional active agent.

The combination of tapinarof with at least one additional AhR activator also cancels or diminishes the tapinarof folliculitis side-effect.

In some embodiments, the effective amount is a therapeutically effective amount of tapinarof and an additional AhR activator or of tapinarof, an additional AhR activator and at least one additional active agent, namely an amount which will cure, treat, alleviate or prevent a skin disorder selected from acne, rosacea, plaque psoriasis, flexural/inverse psoriasis, scalp psoriasis, sebopsoriasis, angular cheilitis, tinea, scabies, scaling skin, dermatitis, erythrasma, folliculitis, external vulvar pruritus and genital psoriasis.

In some embodiments, co-administration of tapinarof and an additional AhR activator or of tapinarof, an additional AhR activator and at least one additional active agent provides an additive and/or synergistic effect while treating, preventing or alleviating a skin disorder.

In some other embodiments, the co-administration may be made either by administration of a single combination composition, or alternatively by separate administration of a first composition comprising tapinarof and an additional AhR activator and a second composition comprising at least one additional active agent.

In another embodiment, each of tapinarof, the additional AhR activator and optionally at least one additional active agent are formulated separately and are administered concomitantly or sequentially.

### Regimen of Administration of the Tapinarof-AhR Activator-Optional Additional Active Agent Topical Combination Compositions

Therapeutically effective concentrations of the active agents in the compositions of this invention for treatment, prevention or alleviation of the symptoms manifested by a skin disorder are determined by empirical methods known in the art.

The concentration of the active agents in the composition will depend on absorption, inactivation, excretion rates of the active compound, synergistic and/or additive effects, the dosage schedule, and amount administered as well as other factors known to those of skill in the art. Generally, the dosages and concentrations will be lower, typically at least about or at 5 to 10% lower but up to about or at 15, 20, 25, 30, 35, 40, 50, 90 or 95% lower than the amount of tapinarof and an additional AhR activator in the developed or marketed single drug currently being developed or used for the treatment of a skin disorder. The dosage and regimen of administration may be determined by dose finding studies, as known in the art.

Exemplary dosages, strengths and concentrations of tapinarof in the topical combination compositions of this invention, are in the range of from about or at 0.1%, 0.25%, 0.5%, 1%, 2% or 3% w/w. Typical strengths in the topical combination compositions of this invention are 0.25%, 0.5% or 1% w/w tapinarof.

Exemplary dosages, strengths and concentrations of the at least one imidazole active agent in the topical combination compositions of this invention, are in the range of from about 0.25% w/w to about 3% w/w or at 0.1%, 0.25%, 0.5%, 1%, 2% or 3% w/w. Typical strengths in the topical combination compositions of this invention are 0.25%, 0.5%, 1% or 2% w/w at least one imidazole active agent.

Exemplary strengths and concentrations of the least one JAK inhibitor in the topical combination compositions are 0.1%, 0.25%, 0.5%, 1%, 2% or 3% w/w. Typical strengths in the topical combination compositions of this invention are 0.1%, 0.25%, 0.5% or 1% w/w.

Exemplary strengths and concentrations of BPO in the topical combination compositions comprising BPO are 2%, 3%, 4%, 5%, 6%, 7%, 8%, 9% or 10% w/w. Typical strengths in the topical combination compositions of this invention are 5%, or 10% w/w.

Exemplary strengths and concentrations of the least one retinoid in the topical combination compositions are 0.01%, 0.25%, 0.05%, 0.1%, 0.2%, 0.3%, 0.4% or 0.5% w/w. Typical strengths in the topical combination compositions of this invention are 0.05%, or 0.11% w/w.

The frequency of administration can be determined empirically. Exemplary frequencies are once daily, twice daily, weekly, bi-weekly or monthly.

Typical administration frequencies of the topical combination compositions of this invention are once daily and twice daily.

Dosage frequencies can be gradually attenuated over time and maintained at a steady dose suitable for long-term—six months, 1 year, 5 years, 10 years or more, up to lifelong administration to control the symptoms of the skin disorder. For example, dosage administration can begin at from twice a day, to once a day, to two times a week, to once a week, to once every two weeks or less frequent than once every two weeks.

Treatment of a Skin Disorder with Combination Compositions Comprising Tapinarof—AhR Activator—Optionally Additional Active Agent

According to another aspect of this invention, skin disorders selected from acne, rosacea, plaque psoriasis, flexural/inverse psoriasis, scalp psoriasis, sebopsoriasis, angular cheilitis, tinea, scabies, scaling skin, dermatitis, erythrasma, folliculitis, external vulvar pruritus and genital psoriasis, are treated with tapinarof combinations with an additional AhR activator and optionally at least one additional active agent.

Due to the synergistic and/or additive effect with tapinarof, the at least one additional active agent can be used at a lower strength than the US-marketed topical drugs used for the treatment of the same medical indication.

### Kits

Kits containing the combination compositions optionally including instructions for administration are provided. The combinations include, for example, the compositions as provided herein. Additionally, provided herein are kits containing the above-described combinations and optionally instructions for administration by topical, transdermal, or other routes, depending on the composition to be delivered.

The compositions provided herein can be packaged as articles of manufacture containing packaging material, a composition provided herein, and a label that indicates that the composition is for treating a skin disorder and is formulated for topical delivery.

The articles of manufacture provided herein contain packaging materials. Packaging materials for use in packaging pharmaceutical products are well known to those of skill in the art. Examples of pharmaceutical packaging materials include, but are not limited to bottles, tubes, containers, application syringes and any packaging material suitable for a selected formulation and intended mode of administration and treatment.

### Embodiments

In some embodiments, there is provided a topical composition comprising from about 0.25% w/w to about 3.0% w/w tapinarof, from about 0.25% w/w to about 3% w/w at least one additional AhR activator and a carrier suitable for topical administration

In some embodiments, there is provided the above topical composition, wherein the at least one AhR activator is selected from an azole active agent, an allylamine active agent, a flavonoid and combinations thereof.

In some embodiments, there is provided a topical composition comprising from about 0.25% w/w to about 3.0% w/w tapinarof, from about 0.25% w/w to about 3% w/w at least one additional AhR activator selected from an azole active agent, an allylamine active agent, a flavonoid and combinations thereof and a carrier suitable for topical administration, wherein the azole active agent is selected from an imidazole selected from ketoconazole, clotrimazole, econazole, tioconazole, miconazole and butoconazole, a triazole selected from itraconazole, fluconazole, terconazole, voriconazole, eficonazole, albaconazole, ravuconazole and isavuconazole and combinations thereof.

In some embodiments, there is provided a topical composition comprising from about 0.25% w/w to about 3.0% w/w tapinarof, from about 0.25% w/w to about 3% w/w at least one additional AhR activator selected from an azole active agent selected from an imidazole and a triazole active agent, an allylamine active agent, a flavonoid and combinations thereof and a carrier suitable for topical administration, wherein said imidazole active agent is from about 0.25% w/w to about 3% w/w ketoconazole. In some embodiments, the imidazole is from about 0.25% w/w to about 3% w/w ketoconazole.

In some embodiments, there is provided a topical composition comprising from about 0.25% w/w to about 3.0% w/w tapinarof, from about 0.25% w/w to about 3% w/w at least one additional AhR activator selected from an azole active agent selected from an imidazole and a triazole active agent, an allylamine active agent, a flavonoid and combinations thereof and a carrier suitable for topical administration, wherein said triazole active agent is from about 0.25% w/w to about 3% w/w itraconazole. In some embodiments, the imidazole is from about 0.25% w/w to about 3% w/w itraconazole.

In some embodiments, there is provided a topical composition comprising from about 0.25% w/w to about 3.0% w/w tapinarof, from about 0.25% w/w to about 3% w/w at least one additional AhR activator selected from an azole active agent selected from an imidazole and a triazole active agent, an allylamine active agent, a flavonoid and combinations thereof and a carrier suitable for topical administration, wherein said allylamine active agent is selected from amorolfine, butenafine, naftifine, terbinafine and combinations thereof.

In some embodiments, the topical composition of the present invention further comprises at least one additional active agent selected from about 0.025% w/w to about 0.5% w/w of at least one retinoid, from about 2% w/w to about 10% w/w benzoyl peroxide (BPO), from about 0.1% w/w to about 3.0% w/w at least one Janus kinase inhibitor (JAK inhibitor), from about 0.01% w/w to about 0.25% w/w at least one corticosteroid of potency class 1-4, from about 0.5% to about 5% w/w at least one acaricide and combinations thereof.

In some embodiments, there is provided a topical composition comprising from about 0.25% w/w to about 3.0% w/w tapinarof, from about 0.25% w/w to about 3% w/w at least one AhR activator selected from an azole active agent is selected from an imidazole selected from ketoconazole, clotrimazole, econazole, tioconazole, miconazole and butoconazole, a triazole selected from itraconazole, fluconazole, terconazole, voriconazole, eficonazole, albaconazole, ravuconazole and isavuconazole and combinations thereof and a carrier suitable for topical administration, further comprising at least one additional active agent selected from about 0.025% w/w to about 0.5% w/w of at least one retinoid, from about 2% w/w to about 10% w/w benzoyl peroxide (BPO), from about 0.1% w/w to about 3.0% w/w at least one Janus kinase inhibitor (JAK inhibitor), from about 0.01% w/w to about 0.25% w/w at least one corticosteroid of potency class 1-4, from about 0.5% to about 5% w/w at least one acaricide and combinations thereof.

In some embodiments, there is provided the above composition comprising tapinarof, at least one additional AhR activator and at least one additional active agent, wherein consisting of two separate compositions, a first composition comprising tapinarof and at least one AhR activator and a second composition comprising at least one additional active agent.

In some embodiments, there is provided the above topical composition comprising tapinarof, at least one additional AhR activator and at least one additional active agent, selected from about 0.025% w/w to about 0.5% w/w of at least one retinoid, from about 2% w/w to about 10% w/w benzoyl peroxide (BPO), from about 0.1% w/w to about 3.0% w/w at least one Janus kinase inhibitor (JAK inhibitor), from about 0.01% w/w to about 0.25% w/w at least one corticosteroid of potency class 1-4, from about 0.5% to about 5% w/w at least one acaricide and combinations thereof, wherein the at least one retinoid is selected from tretinoin, adapalene, tazarotene and combinations thereof.

In some embodiments, there is provided the above topical composition comprising tapinarof, at least one additional AhR activator and at least one additional active agent, selected from about 0.025% w/w to about 0.5% w/w of at least one retinoid, from about 2% w/w to about 10% w/w benzoyl peroxide (BPO), from about 0.1% w/w to about 3.0% w/w at least one Janus kinase inhibitor (JAK inhibitor), from about 0.01% w/w to about 0.25% w/w at least one corticosteroid of potency class 1-4, from about 0.5% to about 5% w/w at least one acaricide and combinations thereof, wherein the at least one JAK inhibitor is selected from tofacitinib, abrocitinib, delgocitinib, ruxolitinib and combinations thereof.

In some embodiments, there is provided the above topical composition comprising tapinarof, at least one additional AhR activator and at least one additional active agent, selected from about 0.025% w/w to about 0.5% w/w of at least one retinoid, from about 2% w/w to about 10% w/w benzoyl peroxide (BPO), from about 0.1% w/w to about 3.0% w/w at least one Janus kinase inhibitor (JAK inhibitor), from about 0.01% w/w to about 0.25% w/w at least one corticosteroid of potency class 1-4, from about 0.5% to about 5% w/w at least one acaricide and combinations thereof, wherein the at least one acaricide is selected from permethrin, ivermectin, crotamiton and combinations thereof.

In some embodiments, there is provided the above topical composition comprising tapinarof, at least one additional AhR activator and at least one additional active agent, selected from about 0.025% w/w to about 0.5% w/w of at least one retinoid, from about 2% w/w to about 10% w/w benzoyl peroxide (BPO), from about 0.1% w/w to about 3.0% w/w at least one Janus kinase inhibitor (JAK inhibitor), from about 0.01% w/w to about 0.25% w/w at least one corticosteroid of potency class 1-4, from about 0.5% to about 5% w/w at least one acaricide and combinations thereof, wherein one or more of the active agents, selected from tapinarof, additional AhR activator and/or additional active agent are encapsulated.

In some embodiments, there is provided a dosage form comprising any of the above compositions of this invention, wherein the composition is formulated in a dosage form selected from a cream, a lotion, a gel, an ointment, an emulsion, a solution, a suspension, an elixir, a tincture, a paste, a foam, an aerosol, a spray, a patch, a transdermal patch, a powder, a shake lotion, a solid, a sponge, a tape, a shampoo and an applicator syringe (for intradermal injection).

In some embodiments, there is provided a method of treatment, prevention or alleviation of a skin disorder, by topical administration to a skin disorder subject in need thereof of a therapeutically effective amount of any of the above compositions of this invention, comprising from about 0.25% w/w to about 3.0% w/w tapinarof, from about 0.25% w/w to about 3% w/w at least one additional AhR activator and optionally at least one additional active agent.

In some embodiments, there is provided a method of treatment, prevention or alleviation of a skin disorder, by topical administration to a skin disorder subject in need thereof of a therapeutically effective amount of any of the above compositions of this invention, comprising from about 0.25% w/w to about 3.0% w/w tapinarof, from about 0.25% w/w to about 3% w/w at least one additional AhR activator selected from an azole active agent selected from an imidazole selected from ketoconazole, clotrimazole, econazole, tioconazole, miconazole and butoconazole, a triazole selected from itraconazole, fluconazole, terconazole, voriconazole, eficonazole, albaconazole, ravuconazole and isavuconazole and combinations thereof and a carrier suitable for topical administration, optionally further comprising at least one additional active agent selected from about 0.025% w/w to about 0.5% w/w of at least one retinoid, from about 2% w/w to about 10% w/w benzoyl peroxide (BPO), from about 0.1% w/w to about 3.0% w/w at least one Janus kinase inhibitor (JAK inhibitor), from about 0.01% w/w to about 0.25% w/w at least one corticosteroid of potency class 1-4, from about 0.5% to about 5% w/w at least one acaricide.

In some embodiments, there is provided the above method, wherein the topical administration is made to the affected skin area of a subject in need thereof as two separate compositions, a first composition comprising tapinarof and said additional AhR activator and a second composition comprising said at least one additional active agent, to be administered from two application syringes or from a dual chamber application syringe, simultaneously or sequentially, in either order and to be mixed on the skin of a subject in need thereof.

In some embodiments, there is provided a method of treatment, prevention or alleviation of a skin disorder, by topical administration to a skin disorder subject in need thereof of a therapeutically effective amount of any of the compositions of this invention, wherein the skin disorder is selected from acne, rosacea, plaque psoriasis, flexural/inverse psoriasis, scalp psoriasis, sebopsoriasis, angular cheilitis, tinea, scabies, scaling skin, dermatitis, erythrasma, folliculitis, external vulvar pruritus and genital psoriasis.

In some embodiments, there is provided a method of treatment, prevention or alleviation of a skin disorder, by topical administration to a skin disorder subject in need thereof of a therapeutically effective amount of any of the compositions of this invention, wherein the skin disorder is acne, selected from acne vulgaris, papulopustular acne and nodular acne.

In some embodiments, there is provided a method of treatment, prevention or alleviation of a skin disorder, by topical administration to a skin disorder subject in need thereof of a therapeutically effective amount of any of the compositions of this invention, wherein the skin disorder is rosacea, selected from erythematotelangiectatic rosacea, papulopustular rosacea, rhinophyma rosacea and ocular rosacea.

In some embodiments, there is provided a method of treatment, prevention or alleviation of a skin disorder, by topical administration to a skin disorder subject in need thereof of a therapeutically effective amount of any of the compositions of this invention, wherein the skin disorder is dermatitis (eczema), selected from atopic dermatitis, contact dermatitis, dyshidrotic dermatitis, nummular dermatitis and seborrheic dermatitis.

In some embodiments, there is provided a method of treatment, prevention or alleviation of a skin disorder, by topical administration to a skin disorder subject in need thereof of a therapeutically effective amount of any of the compositions of this invention, wherein the skin disorder is tinea, selected from tinea pedis, tinea capitis, tinea barbae, tinea faciei, tinea corporis, tinea manum, tinea cruris, tinea unguium (onychomycosis) and tinea interdigital.

In some embodiments, there is provided a method of treatment, prevention or alleviation of a skin disorder, by topical administration to a skin disorder subject in need thereof of a therapeutically effective amount of any of the compositions of this invention, wherein the skin disorder is selected from acne, rosacea, plaque psoriasis, flexural/inverse psoriasis, scalp psoriasis, sebopsoriasis, angular cheilitis, tinea, scabies, scaling skin, dermatitis, erythrasma, folliculitis, external vulvar pruritus and genital psoriasis, wherein the treatment comprises once daily or twice daily topical administration to a skin disorder subject in need thereof of a therapeutically effective amount of the composition of this invention comprising from about 0.25% w/w to about 3.0% w/w tapinarof, from about 0.25% w/w to about 3% w/w at least one additional AhR activator an azole active agent selected from an imidazole selected from ketoconazole, clotrimazole, econazole, tioconazole, miconazole and butoconazole, a triazole selected from itraconazole, fluconazole, terconazole, voriconazole, eficonazole, albaconazole, ravuconazole and isavuconazole and combinations thereof and a carrier suitable for topical administration.

In some embodiments, there is provided a method wherein the treatment comprises once daily or twice daily topical administration to a skin disorder subject in need thereof of a therapeutically effective amount of the composition comprising from about 0.25% w/w to about 3.0% w/w tapinarof, from about 0.25% w/w to about 3% w/w at least one additional AhR activator selected from an azole active agent selected from an imidazole selected from ketoconazole, clotrimazole, econazole, tioconazole, miconazole and butoconazole, a triazole selected from itraconazole, fluconazole, terconazole, voriconazole, eficonazole, albaconazole, ravuconazole and isavuconazole and combinations thereof, at least one additional active agent selected from about 0.025% w/w to about 0.5% w/w of at least one retinoid, from about 2% w/w to about 10% w/w benzoyl peroxide (BPO), from about 0.1% w/w to about 3.0% w/w at least one Janus kinase inhibitor (JAK inhibitor), from about 0.01% w/w to about 0.25% w/w at least one corticosteroid of potency class 1-4, from about 0.5% to about 5% w/w at least one acaricide and a carrier suitable for topical administration.

In some embodiments, there is provided the above method, wherein tapinarof, the at least one additional AhR activator and/or the at least one additional active agent exhibit an additive or synergistic effect, thereby allowing to reduce the amounts of the active agents in the composition.

In some embodiments, there is provided a method of treatment, prevention or alleviation of a skin disorder, by topical administration to a skin disorder subject in need thereof of a therapeutically effective amount of any of the above compositions of this invention, comprising from about 0.25% w/w to about 3.0% w/w tapinarof, from about 0.25% w/w to about 3% w/w at least one additional AhR activator selected from an azole active agent selected from an imidazole selected from ketoconazole, clotrimazole, econazole, tioconazole, miconazole and butoconazole, a triazole selected from itraconazole, fluconazole, terconazole, voriconazole, eficonazole, albaconazole, ravuconazole and isavuconazole and combinations thereof and a carrier suitable for topical administration, optionally further comprising at least one additional active agent selected from about 0.025% w/w to about 0.5% w/w of at least one retinoid, from about 2% w/w to about 10% w/w benzoyl peroxide (BPO), from about 0.1% w/w to about 3.0% w/w at least one Janus kinase inhibitor (JAK inhibitor), from about 0.01% w/w to about 0.25% w/w at least one corticosteroid of potency class 1-4, from about 0.5% to about 5% w/w at least one acaricide, wherein said at least one additional AhR activator, cancels or diminishes the tapinarof folliculitis side-effect.

In some embodiments, there is provided a regimen of administration comprising the once daily or twice daily administration to a skin disorder subject in need thereof of a therapeutically effective dose of a composition comprising from about 0.25% w/w to about 3.0% w/w tapinarof, from about 0.25% w/w to about 3% w/w at least one additional AhR activator selected from an azole active agent selected from an imidazole selected from ketoconazole, clotrimazole, econazole, tioconazole, miconazole and butoconazole, a triazole selected from itraconazole, fluconazole, terconazole, voriconazole, eficonazole, albaconazole, ravuconazole and isavuconazole and combinations thereof and a carrier suitable for topical administration, until the skin disorder is cured, prevented or alleviated.

In some embodiments, there is provided a regimen of administration comprising the once daily or twice daily administration to a skin disorder subject in need thereof of a therapeutically effective dose of a composition comprising from about 0.25% w/w to about 3.0% w/w tapinarof, from about 0.25% w/w to about 3% w/w at least one additional AhR activator selected from an azole active agent selected from an imidazole selected from ketoconazole, clotrimazole, econazole, tioconazole, miconazole and butoconazole, a triazole selected from itraconazole, fluconazole, terconazole, voriconazole, eficonazole, albaconazole, ravuconazole and isavuconazole and combinations thereof, at least one additional active agent selected from about 0.025% w/w to about 0.5% w/w of at least one retinoid, from about 2% w/w to about 10% w/w benzoyl peroxide (BPO), from about 0.1% w/w to about 3.0% w/w at least one Janus kinase inhibitor (JAK inhibitor), from about 0.01% w/w to about 0.25% w/w at least one corticosteroid of potency class 1-4, from about 0.5% to about 5% w/w at least one acaricide and a carrier suitable for topical administration, until the skin disorder is cured, prevented or alleviated.

In some embodiments, there is provided a regimen of administration comprising the once daily or twice daily administration to a skin disorder subject in need thereof a therapeutically effective amount of a dosage form of this invention.

In some embodiments, there is provided a kit comprising one or more dosage forms of this invention and instructions for use.

### Definitions

Unless defined otherwise, all technical and scientific terms used herein have the same meaning as is commonly understood by one of skill in the art to which the invention pertains. In case of conflict, the specification, including definitions, takes precedence. All patents, patent applications, published applications, articles, publications and other published materials referred to throughout the entire disclosure herein, unless noted otherwise, are incorporated by reference in their entirety.

As used herein, the indefinite articles “a” and “an” mean “at least one” or “one or more” unless the context clearly dictates otherwise.

As used herein, the term “treating” or “treatment” includes curing a condition, treating a condition, preventing a condition, treating symptoms of a condition, curing symptoms of a condition, ameliorating symptoms of a condition, treating effects of a condition, ameliorating effects of a condition, and preventing results of a condition.

As used herein, the terms “pharmaceutically active agent” or “active agent” or “active pharmaceutical ingredient” or “API” are interchangeable and mean the ingredient is a pharmaceutical drug which is biological active and is regulatory approved or approvable as such.

The term “ingredient” refers to a pharmaceutically acceptable ingredient which is included or is amenable to be included in FDA's Inactive Ingredient database (IIG). Inactive ingredients sometimes exhibit some therapeutic effects, although they are not drugs.

As used herein, a “pharmaceutical composition” refers to a composition comprising one or more active ingredients with other components such as pharmaceutically acceptable ingredients or excipients. The purpose of a pharmaceutical composition is to facilitate administration of an active ingredient to a subject.

As used herein, the term “essentially free” generally refers to a composition having less than about 2 percent by weight, more preferably 1 percent per weight, less than about 0.5 percent by weight or even less than 0.1 percent by weight of a certain ingredient, based on the total weight of the composition.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “10 μm” is intended to mean “about 10 μm”.

As used herein, numerical ranges preceded by the term “about” should not be considered to be limited to the recited range. Rather, numerical ranges preceded by the term “about” should be understood to include a range accepted by those skilled in the art for any given element in formulations according to the present invention.

As used herein, when a numerical value is preceded by the term “about”, the term “about” is intended to indicate+/−10%.

The terms “comprise”, “comprising”, “includes”, “including”, “having” and their conjugates mean “including but not limited to”.

The term “consisting of” means “including and limited to”.

The term “consisting essentially of” means that the composition, method formulation may include additional ingredients, steps and/or parts, but only if the additional ingredients, steps and/or parts do not materially alter the basic and novel characteristics of the claimed composition, method or structure.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### Examples

Reference is now made to the following examples, which together with the above descriptions illustrate some embodiments of the invention in a non-limiting fashion.

Generally, the nomenclature used herein, and the laboratory procedures utilized in the present invention include chemical, molecular and biochemical, techniques. Such techniques are thoroughly explained in the literature. General references are provided throughout this document

The procedures therein are believed to be well known in the art and are provided for the convenience of the reader. All the information contained therein is incorporated herein by reference.

In the examples below, all % values referring to a solution are in (w/w).

All % values, referring to dispersions (suspensions) are in (w/w).

Unless otherwise indicated, all solutions used in the example below refer to an aqueous solution of the indicated ingredient.

Tables 1-2 and compositions 1-20 disclosed above-provide exemplary active agents' combination of active agent classes and specific active agents of this invention.

All the exemplary combinations of specific active agents comprise micronized tapinarof, micronized additional AhR activator selected from an azole active agent selected from an imidazole selected from ketoconazole, clotrimazole, econazole, tioconazole, miconazole and butoconazole, a triazole selected from itraconazole, fluconazole, terconazole, voriconazole, eficonazole, albaconazole, ravuconazole and isavuconazole and combinations thereof and optionally an additional active agent selected from a retinoid (selected from tretinoin, adapalene and tazarotene), benzoyl peroxide (BPO), a JAK inhibitor (selected from tofacitinib, abrocitinib, delgocitinib and ruxolitinib), a corticosteroid (selected from potency groups 1-4, e.g. halobetasol) an acaricide (selected from permethrin, ivermectin and crotamiton) and combinations thereof. The strength ranges of all the active agents in the exemplary compositions are detailed in Table 3, comprising 20 exemplary combination compositions.

Tapinarof in the exemplary compositions may be used as such, as detailed in Examples 8-10, or encapsulated as detailed in Examples 1-7. The other active agents in the combination compositions (an additional AhR activator and optionally an additional active agent) may also be used as such, or encapsulated by a similar process, as detailed in Examples 3-7.

### Example 1: Preparation of Silicone Dioxide (SiO₂) Encapsulated Tapinarof Dispersed in Water (15% w/w Tapinarof)

### Preparation of Tapinarof Dispersion and Acid Cocktail

Tapinarof dispersion is prepared by mixing 378 grams of CTAC CT-429 (Cetrimonium Chloride 30%), 6,756 grams of micronized tapinarof having an average particle size of less than 1 μm, and 18,855 grams water under high shear. The dispersion is homogenized for 60 min at 33° C. (no more than 45° C.).

An acid cocktail is prepared using 1013 grams Hydrochloric Acid (37%), 215.3 grams anhydrous Citric Acid, 322.3 grams Lactic Acid (90%), and 1632 grams water.

### Coating Cycle

The coating cycle is started by adding 953 grams sodium silicate solution extra pure (28%) to the tapinarof dispersion prepared in step a) under high shear, followed by adding the acid cocktail prepared in step (a) and followed by adding 1675 grams PDAC (3%) solution to the mixture. The cycle is repeated another 5 times. After the 6 cycles, the pH of the mixture is adjusted to 5.0 using the acid cocktail, and water is added to complete the total weight of the mixture to 45 kilograms.

The composition of the final tapinarof water suspension product is shown in Table 3.

TABLE 3
Composition of the encapsulated tapinarof 15% water suspension

| Ingredient | % w/w of ingredient in the suspension |
|---|---|
| Polyquarternium-7 | 5.6 |
| Hydrochloric Acid 37% | 2.0 |
| Citric Acid, Anhydrous | 0.4 |
| Lactic Acid | 0.6 |
| Silicone Dioxide | 3.4 |
| Sodium Hydroxide | 1.4 |
| Cetrimonium Chloride | 0.84 |
| Tapinarof | 15.00 |
| Sterile Water for Irrigation | Up to 100% |

### Example 2

### Preparation of Encapsulated Tapinarof Dispersed in Oil (3.06% w/w Tapinarof)

### a) Oil Phase

45.9 grams of micronized tapinarof having an average particle size of less than 1 μm are mixed in 129.3 grams of Squalane. 86.16 grams of Tetraethoxysilane (TEOS) are added, and the resulted mixture was milled at 5000 rpm in a ball mill for 10 minutes with an upper propeller mixer at a speed of 250 rpm for 7 minutes, followed by 400 rpm for 3 minutes. 140.4 grams of milled tapinarof in oil is aliquoted out and then heated to 60° C. 9.0 grams of Beeswax are added and melted in the oil phase.

### b) Water Phase

3.3 grams CTAC (Cetrimonium Chloride) are dissolved in 490.0 g water at 60° C. Unless indicated otherwise, in all examples described herein, the term “water” refers to sterile water for irrigation (USP).

### c) Core-Shell Step

124.5 grams of the oil phase prepared in step (a) is added to the water phase and homogenized at 4000 rpm for 1 minute. 17.9 grams of sodium silicate extra pure solution (28%) are added to the emulsion. The pH of the emulsion is adjusted to 4.0 using HCl 5N solution. Water is added to complete the total weight of the mixture to 650 grams. The suspension is then stirred for 17 hours at 25° C. for the TEOS hydrolysis to be completed. The composition of the final encapsulated tapinarof water suspension product is shown in Table 4.

TABLE 4
Composition of the encapsulated tapinarof 3.06% water suspension

| Ingredient | % of pure ingredient in the suspension |
|---|---|
| Beeswax | 1.15 |
| Squalane | 8.62 |
| Tetraethoxysilane | 5.74 |
| Tapinarof | 3.06 |
| Cetrimonium Chloride | 0.15 |
| Sodium hydroxide | 0.74 |
| Hydrochloric acid | 0.40 |
| Sterile Water for Irrigation | Up to 100% |

### Example 3: Preparation of Encapsulated Itraconazole Dispersed in Oil (3.06% w/w Itraconazole)

### a) Oil Phase

45.9 grams of itraconazole are mixed in 129.3 grams of Squalane. 86.16 grams of Tetraethoxysilane (TEOS) are added, and the resulted mixture was milled at 5000 rpm in a ball mill for 10 minutes with an upper propeller mixer at a speed of 250 rpm for 7 minutes, followed by 400 rpm for 3 minutes. 140.4 grams of milled itraconazole in oil is aliquoted out and then heated to 60° C. 9.0 grams of Beeswax are added and melted in the oil phase.

### b) Water Phase

3.3 grams CTAC (Cetrimonium Chloride) are dissolved in 490.0 g water at 60° C. Unless indicated otherwise, in all examples described herein, the term “water” refers to sterile water for irrigation (USP).

### c) Core-Shell Step

124.5 grams of the oil phase prepared in step (a) is added to the water phase and homogenized at 4000 rpm for 1 minute. 17.9 grams of sodium silicate extra pure solution (28%) are added to the emulsion. The pH of the emulsion is adjusted to 4.0 using HCl 5N solution. Water is added to complete the total weight of the mixture to 650 grams. The suspension is then stirred for 17 hours at 25° C. for the TEOS hydrolysis to be completed. The composition of the final encapsulated itraconazole water suspension product is shown in Table 5.

TABLE 5
Composition of the encapsulated itraconazole
3.06% water suspension

| Ingredient | % of pure ingredient in the suspension |
|---|---|
| Beeswax | 1.15 |
| Squalane | 8.62 |
| Tetraethoxysilane | 5.74 |
| Itraconazole | 3.06 |
| Cetrimonium Chloride | 0.15 |
| Sodium hydroxide | 0.74 |
| Hydrochloric acid | 0.40 |
| Sterile Water for Irrigation | Up to 100% |

### Example 4: Preparation of a Combination Composition Comprising 1.5% w/w Encapsulated Tapinarof—1.5% w/w Encapsulated Itraconazole

The topical 1.5% w/w encapsulated tapinarof—1.5% w/w encapsulated itraconazole combination composition is obtained by intimately mixing 500 g of the composition of Example 2 (3.06% tapinarof) with 500 g of the composition of Example 3 (3.06% itraconazole) until a homogeneous mixture is obtained.

1000 g of a combination composition comprising 1.5% w/w encapsulated tapinarof and 1.5% w/w encapsulated itraconazole is obtained.

### Example 5: Preparation of Encapsulated BPO 5% w/w Cream

Oil Phase: 720.0 of grams Cyclomethicone 5-N, 540.0 of grams Cetyl Alcohol, 360.0 grams Polyoxyl 100 Stearate, and 540.0 grams of Glyceryl Monostearate were mixed at 70° C.

Water phase: 18.0 grams of ethylenediamine tetraacetate Disodium salt were dissolved in 6500 grams of water. 720.0 grams of glycerin (99.5%) were added to the solution. After the solution was heated to 70° C., 72.0 grams of Carbopol 980 NF were added and the resulting mixture was homogenized at 3300 rpm for 10 minutes to ensure that all materials completely melted and dissolved. 76.5 grams if sodium hydroxide (20%) were then added and the mixture was stirred under high shear for 10 minutes at no less than 70° C.

The oil phase was added to the water phase under high shear at 78° C., and the resulting emulsion was homogenized at 3300 rpm for 10 minutes. 72.0 grams of Citric Acid and 6000 grams of benzoyl peroxide (BPO) 15% water suspension made as described below were mixed. The resulting mixture was added to the emulsion at 65° C. and mixed at 1400 rpm for 10 minutes. The emulsion was cooled to 35° C. and the pH of the emulsion was adjusted to 3.6 using HCl 5N solution and then water was added until the total weight of the emulsion reached 18 kilograms.

### Preparation of a 15% BPO Water Suspension

A benzoyl peroxide (BPO) suspension was prepared by mixing 125.67 grams of CTAC CT-429 (Cetrimonium Chloride 30%), 3000 grams of hydrous benzoyl peroxide, and 5200 g water under high shear. The suspension was homogenized for 60 minutes at 33° C. (no more than 45° C.), and then the pH of the solution was adjusted to 7.0 using sodium hydroxide solution (20%). An acid cocktail was prepared using 493 grams hydrochloric acid (37%), 98 grams anhydrous Citric Acid, 147 grams Lactic Acid (90%), and 794 grams water.

### Stabilization of the 15% BPO Water Suspension

Stabilization of the suspension was done by adding 532 grams of silicon dioxide to the benzoyl peroxide suspension prepared in the above step under high shear, followed by adding 855 grams 10% PDAC (Polyquarternium-7) solution to the mixture. The mixture was stirred under high shear for 2 hours. The pH of the mixture was adjusted to 5.0 using the above acid cocktail, and water was added to complete the total weight of the mixture to 15 kilograms.

### Example 6: Preparation of a Triple Encapsulated Combination Composition Comprising 1% w/w Encapsulated Tapinarof, 1% w/w Encapsulated Itraconazole and 5% Encapsulated BPO

The triple encapsulated tapinarof-itraconazole-BPO combination composition is prepared by intimately mixing 670 g of the composition of Example 4 combination composition comprising 1.5% w/w encapsulated tapinarof+1.5% w/w encapsulated itraconazole and 416 g of the composition of Example 8 comprising an encapsulated 15% w/w BPO composition, affording 1086 g of the triple encapsulated tapinarof-itraconazole-BPO combination composition comprising 1% w/w encapsulated tapinarof, 1% w/w encapsulated itraconazole and 5% w/w encapsulated BPO.

### Example 7: Preparation of a Triple Encapsulated Combination Composition for Administration as Two Separate Compositions to be Mixed on the Skin of a Subject to Provide a Combination Composition Comprising 1% w/w Encapsulated Tapinarof, 1% w/w Encapsulated Itraconazole and 5% Encapsulated BPO

The topical tapinarof-itraconazole-BPO combination composition of this Example consists of two separate compositions 1 and 2, to be administered to the affected skin area of a subject in need thereof as two separate compositions. from two application syringes or a dual chamber application syringe, simultaneously or sequentially in either order. The two separate compositions 1 and 2 are to be mixed on the skin of a subject in need thereof, to provide a combination composition comprising 1% w/w encapsulated tapinarof, 1% w/w encapsulated itraconazole and 5% encapsulated BPO.

The dual chamber dispensing system used is of the type described in EP-A-0644129 and U.S. Pat. No. 5,356,040, the contents of which are incorporated herein by reference. Such a system has two side-by-side chambers, each equipped with a dispensing valve; these are operated by adjacent actuators so as to dispense the formulations either simultaneously or separately as desired. Suitable dispensing systems. having chambers which are each capable of holding about 15 ml of composition, are available from Maplast S. r. 1., Via Pasublo 3, Tradate 21049 VA, Italy. The respective dimensions of the dispenser means may be chosen to provide dispensing of the respective compositions in a predetermined ratio.

### Composition 1

15 g of the composition of Example 4 combination composition comprising 1.5% w/w encapsulated tapinarof-1.5% w/w encapsulated itraconazole is to be charged in one of two application syringes or in one of the two chambers of a dual chamber application syringe.

### Composition 2

9.3 g of the composition of Example 8 comprising an encapsulated 15% w/w BPO composition is to be charged in one of two application syringes or in one of the two chambers of a dual chamber application syringe.

### Administration of Compositions 1 and 2

The two compositions 1 and 2 are to be mixed on the skin of a subject in need thereof in the above 15:9.3 (1.61) ratio, affording a total of 24.3 g of the triple composition, to be applied to the affected area of the skin of a subject in several applications, each application comprising a therapeutically effective amount of the triple combination comprising 1% w/w encapsulated tapinarof, 1% w/w encapsulated itraconazole and 5% w/w encapsulated BPO.

### Example 8: Preparation of a Non-Encapsulated Tapinarof-Itraconazole Combination Composition

The topical tapinarof-itraconazole combination cream consists of:

0.25-3.0% w/w tapinarof,
0.25-3.0% w/w itraconazole
0.1-0.5% w/w menthol,
0.01-0.05% w/w butylated hydroxyanisole (BHA),
15-30% w/w propylene glycol,
5.0-15.0% polysorbate 80,
10-25% w/w glyceryl monostearate,
10-25% w/w of thickener octadecanol,
Adjust to pH 6.0-7.0 with 0.1M NaOH or 0.1M HCl

The cream composition is prepared by the following steps:

(1) weigh tapinarof having an average particle size of less than 1 μm;

(2) heat the propylene glycol to 60° C. in a water bath;

(3) add to the heated propylene glycol while stiffing tapinarof, itraconazole, BHT, menthol, octadecanol, polysorbate 80 and glyceryl monostearate, and dissolve to obtain an oil phase;

(4) prepare the aqueous phase by heating purified water in a water bath to 60° C., stir in and dissolve polysorbate 80, make up to 100% with purified water and adjust the pH to 6.0-7.0 with 0.1 M NaOH or HCl;

(5) add the aqueous phase to the oil phase under vacuum stirring, and cool to room temperature to obtain a cream;

(6) fill the tapinarof-itraconazole combination cream in an aluminum tube or other delivery system.

### Example 9: Preparation of a Non-Encapsulated Tapinarof-Ketoconazole Combination Composition

The topical tapinarof-ketoconazole combination cream consists of:

0.25-3.0% w/w tapinarof,
0.25-3.0% w/w ketoconazole,
0.1-0.5% w/w menthol,
0.01-0.05% w/w butylated hydroxyanisole (BHA),
15-30% w/w propylene glycol,
5.0-15.0% polysorbate 80,
10-25% w/w glyceryl monostearate,
10-25% w/w of thickener octadecanol,
Adjust to pH 6.0-7.0 with 0.1M NaOH or 0.1M HCl,

The cream composition is prepared by the following steps:

(1) weigh tapinarof having an average particle size of less than 1 μm;

(2) heat the propylene glycol to 60° C. in a water bath;

(3) add to the heated propylene glycol while stirring tapinarof, ketoconazole, BHT, menthol, octadecanol, polysorbate 80 and glyceryl monostearate, and dissolve to obtain an oil phase;

(4) prepare the aqueous phase by heating purified water in a water bath to 60° C., stir in and dissolve polysorbate 80, make up to 100% with purified water and adjust the pH to 6.0-7.0 with 0.1 M NaOH or HCl;

(5) add the aqueous phase to the oil phase under vacuum stirring, and cool to room temperature to obtain a cream;

(6) fill the tapinarof-ketoconazole combination cream in an aluminum tube or other delivery system.

### Example 10: Preparation of a Non-Encapsulated Tapinarof-Itraconazole-Tretinoin Triple Combination Composition

The topical tapinarof-itraconazole-tretinoin triple combination cream consists of:

0.25-3.0% w/w tapinarof,
0.25-3.0% w/w itraconazole,
0.025%-0.5% w/w tretinoin,
0.1-0.5% w/w menthol,
0.01-0.05% w/w butylated hydroxyanisole (BHA),
15-30% w/w propylene glycol,
5.0-15.0% polysorbate 80,
10-25% w/w glyceryl monostearate,
10-25% w/w of thickener octadecanol,
Adjust to pH 6.0-7.0 with 0.1M NaOH or 0.1M HCl.

The cream composition is prepared by the following steps:

(1) weigh tapinarof having an average particle size of less than 1 μm;

(2) heat the propylene glycol to 60° C. in a water bath;

(3) add to the heated propylene glycol while stiffing tapinarof, itraconazole, tretinoin, BHT, menthol, octadecanol, polysorbate 80 and glyceryl monostearate, and dissolve to obtain an oil phase;

(4) prepare the aqueous phase by heating purified water in a water bath to 60° C., stir in and dissolve polysorbate 80, make up to 100% with purified water and adjust the pH to 6.0-7.0 with 0.1 M NaOH or HCl;

(5) add the aqueous phase to the oil phase under vacuum stirring, and cool to room temperature to obtain a cream;

(6) fill the tapinarof-itraconazole-tretinoin combination cream in an aluminum tube or other delivery system.

### Examples 11-30: Preparation of Encapsulated Tapinarof-Imidazole and Optionally Additional Active Agent Combination Compositions

The exemplary compositions 1-20 of Table 1 are prepared in encapsulated form by using the proper process selected from Examples 1-7.

### Examples 31-50: Preparation of Non-Encapsulated Tapinarof-Imidazole and Optionally Additional Active Agent Combination Compositions

The exemplary compositions 1-20 of Table 1 are prepared in non-encapsulated form by using the proper process selected from Examples 8-10.

1. A topical composition comprising from about 0.25% w/w to about 3.0% w/w tapinarof, from about 0.25% w/w to about 3% w/w at least one additional AhR activator and a carrier suitable for topical administration.

2. The composition of claim 1, wherein the at least one AhR activator is selected from an azole active agent, an allylamine active agent, a flavonoid and combinations thereof.

3. The composition of claim 2, wherein the azole active agent is selected from an imidazole selected from ketoconazole, clotrimazole, econazole, tioconazole, miconazole and butoconazole, a triazole selected from itraconazole, fluconazole, terconazole, voriconazole, eficonazole, albaconazole, ravuconazole and isavuconazole and combinations thereof.

4. The composition of claim 3, wherein the imidazole is from about 0.25% w/w to about 3% w/w ketoconazole.

5. The composition of claim 3, wherein the triazole is from about 0.25% w/w to about 3% w/w itraconazole.

6. The composition of claim 2, wherein the allylamine active agent is selected from amorolfine, butenafine, naftifine, terbinafine and combinations thereof.

7. The composition of claim 1, further comprising at least one additional active agent selected from about 0.025% w/w to about 0.5% w/w of at least one retinoid, from about 2% w/w to about 10% w/w benzoyl peroxide (BPO), from about 0.1% w/w to about 3.0% w/w at least one Janus kinase inhibitor (JAK inhibitor), from about 0.01% w/w to about 0.25% w/w at least one corticosteroid of potency class 1-4, from about 0.5% to about 5% w/w at least one acaricide and combinations thereof.

8. The composition of claim 7, wherein said composition consisting of two separate compositions, a first composition comprising tapinarof and at least one AhR activator and a second composition comprising at least one additional active agent.

9. The composition of claim 7, wherein the at least one retinoid is selected from tretinoin, adapalene, tazarotene and combinations thereof.

10. The composition of claim 7, wherein the at least one JAK inhibitor is selected from tofacitinib, abrocitinib, delgocitinib, ruxolitinib and combinations thereof.

11. The composition of claim 7, wherein the at least one acaricide is selected from permethrin, ivermectin, crotamiton and combinations thereof.

12. The composition of claim 1, wherein at least one of the active agents tapinarof, or at least one AhR activator is encapsulated.

13. A dosage form comprising the composition of claim 1, wherein the composition is formulated in a dosage form selected from a cream, a lotion, a gel, an ointment, an emulsion, a solution, a suspension, an elixir, a tincture, a paste, a foam, an aerosol, a spray, a patch, a transdermal patch, a powder, a shake lotion, a solid, a sponge, a tape, a shampoo and an applicator syringe (for intradermal injection).

14. A method of treatment, prevention or alleviation of a skin disorder, by topical administration to the affected area of a skin disorder subject in need thereof of a therapeutically effective amount of a composition of claim 1, and optionally at least one additional active agent.

15. The method of claim 14, wherein the topical administration is made to the affected skin area of a subject in need thereof as two separate compositions, a first composition comprising tapinarof and said at least one AhR activator and a second composition comprising said at least one additional active agent, to be administered from two application syringes or from a dual chamber application syringe, simultaneously or sequentially in either order, wherein the two compositions are mixed on the skin of a subject in need thereof.

16. The method of claim 14, wherein the skin disorder is selected from acne, rosacea, plaque psoriasis, flexural/inverse psoriasis, scalp psoriasis, sebopsoriasis, angular cheilitis, tinea, scabies, scaling skin, dermatitis, erythrasma, folliculitis, external vulvar pruritus and genital psoriasis.

17. The method of claim 16, wherein the skin disorder is acne, selected from acne vulgaris, papulopustular acne and nodular acne.

18. The method of claim 16, wherein the skin disorder is rosacea, selected from erythematotelangiectatic rosacea, papulopustular rosacea, rhinophyma rosacea and ocular rosacea.

19. The method of claim 16, wherein the skin disorder is dermatitis (eczema), selected from atopic dermatitis, contact dermatitis, dyshidrotic dermatitis, nummular dermatitis and seborrheic dermatitis.

20. The method of claim 16, wherein the skin disorder is tinea, selected from tinea pedis, tinea capitis, tinea barbae, tinea faciei, tinea corporis, tinea manum, tinea cruris, tinea unguium (onychomycosis) and tinea interdigital.

21. The method of claim 14, wherein the treatment comprises once daily or twice daily topical administration to a skin disorder subject in need thereof of a therapeutically effective amount of said composition.

22. The method of claim 14, wherein tapinarof and the at least one imidazole active agent and/or the at least one additional active agent exhibit an additive or synergistic effect, thereby allowing to reduce the amounts of the active agents in the composition.

23. The method of claim 14, wherein said at least one additional AhR activator, cancels or diminishes the tapinarof folliculitis side-effect.

24. A regimen of administration comprising once daily or twice daily administration to a skin disorder subject in need thereof of a therapeutically effective dose of the composition of claim 1 until the skin disorder is cured, prevented or alleviated.

25. A regimen of administration comprising once daily or twice daily administration to a skin disorder subject in need thereof a therapeutically effective amount of the dosage form of claim 13.

26. A kit comprising one or more dosage forms of claim 13 and instructions for use.

27. The composition of claim 7, wherein at least one of the active agents tapinarof, at least one AhR activator or at least one additional active agent is encapsulated.

28. A dosage form comprising the composition of claim 7, wherein the composition is formulated in a dosage form selected from a cream, a lotion, a gel, an ointment, an emulsion, a solution, a suspension, an elixir, a tincture, a paste, a foam, an aerosol, a spray, a patch, a transdermal patch, a powder, a shake lotion, a solid, a sponge, a tape, a shampoo and an applicator syringe (for intradermal injection).
